(19) United States
(12) Patent Application Publication
(10) Pub. No.: US 2013/0018183 A1
(43) Pub. Date: Jan. 17, 2013

(54) METHOD AND A DEVICE FOR MAKING REDUCING SUGAR

(71) Applicants: Alex Chen-Chang Chang, Taichung (TW); Chiu-Yue Lin, Taichung (TW); Ying-Hsuan Tu, Taichung (TW); Ming-Hsiang Huang, Taichung (TW)

(72) Inventors: Alex Chen-Chang Chang, Taichung (TW); Chiu-Yue Lin, Taichung (TW); Ying-Hsuan Tu, Taichung (TW); Ming-Hsiang Huang, Taichung (TW)

(21) Appl. No.: 13/181,717

(22) Filed: Jul. 13, 2011

(51) Int. Cl. C07H 1/08; B01J 8/00; C07H 1/06

(52) U.S. Cl. 536/128; 536/127; 422/187

(57) ABSTRACT

A method and a device for making reducing sugar firstly combine a bio-substance containing lignocellulose with a solvent, thereby conducting a degradation reaction upon afore mixture through an autoclave so as to promote the bio-substance to solvent ratio during the production. Thus, a reducing sugar solution of a high concentration is produced. In the meantime, the bio-substance containing lignocellulose does not need a high-temperatured steaming after the degradation. Therefore, the water amount and the energy resources that are applied in the present invention are less than those of the conventional technique. Further, the autoclave is a common apparatus in the market, which facilitates in an easy purchase, decreases the producing cost, and benefits the product promotion.

### BACKGROUND OF THE INVENTION

1. Field of the Invention

The present invention relates to a method and a device for making reducing sugar from lignocellulose.

2. Description of the Related Art

In view of the daily reducing petroleum energy resource, novel renewable energies such as solar energy, wind power, waterpower, geothermal energy resource, and biomass energy are developed progressively. In above energy resources, the biomass energy must be one of the most energy resources since the material for making the same could be easily acquired, so there are increasing developments of the biomass energy. In the biomass energy, the bio-ethanol is produced through fermentation of a carbohydrate. Wherein, the carbohydrate is usually extracted from the starche and the sugars. However, the food coordination of supply and demand would vanish while a large amount of starches and sugars are used for fuels but food. Therefore, today's carbohydrate for producing the bio-ethanol should adopt the reducing sugar that is extracted from hardwood, cork, or bio-substance containing lignocellulose. In the existing technique for making the reducing sugar, the bio-substance containing lignocellulose are extruded and mixed with acid by a twin screw extruder. Thereafter, the bio-substance is steamed via a heating temperature so as to conduct hot water leaching. Herein, the reducing sugar in the bio-substance containing lignocellulose is leached out. Nonetheless, afore technique for making the reducing sugar has to adopt the steam of 130° C., and then conduct the steaming on the bio-substance containing lignocellulose that is induced by degradation for 20 minutes. Obviously, a lot of water and energy resources are used, which is wasteful and adverse for the environment protection. Moreover, the twin screw extruder is actually a special device which is difficult to be purchased and the constructing cost thereof is also high. Therefore, the existing technique is unbeneficial for promotion.

### SUMMARY OF THE INVENTION

The present invention is to provide a method and a device for making the reducing sugar that is of low resource wastage and high efficiency.

A method for making reducing sugar in accordance with afore object comprises steps of combining a bio-substance containing lignocellulose with a solvent, and conducting a degradation reaction via an autoclave for acquiring a reducing sugar solution of a high concentration.

A device for making reducing sugar in accordance with afore object comprises an autoclave for accommodating a bio-substance containing lignocellulose and a solvent and for inducing a degradation reaction of the bio-substance containing lignocellulose under a high temperature and a high pressure; and a dehydrator is disposed for dehydrating a product resulted from the degradation reaction so as to separate a reducing sugar solution of a high concentration from solid bio-remains.

Accordingly, the method and the device for making the reducing sugar in the present invention are able to promote the bio-substance to solvent ratio of the reducing sugar solution in the procedure. Thereby, the reducing sugar solution of a high concentration could be acquired. Moreover, the bio-substance containing lignocellulose does not have to be steamed with a high temperature after the degradation. Thus, the present invention utilizes less water and fewer energy resources than the conventional technique. Further, the autoclave and the dehydrator are common apparatus in the market. Therefore, the present invention preferably is advantageous to purchase the apparatus, decrease the producing cost, and achieve an easy promotion.

### BRIEF DESCRIPTION OF THE DRAWINGS

FIG. 1 is a flowchart showing how to make the reducing sugar solution of the present invention;

FIG. 2 is a schematic view showing the correlations between elements in the device of the present invention; and

FIG. 3 is a flow chart showing how to treat the reducing sugar solution of the present invention.

### DETAILED DESCRIPTION OF THE PREFERRED EMBODIMENTS

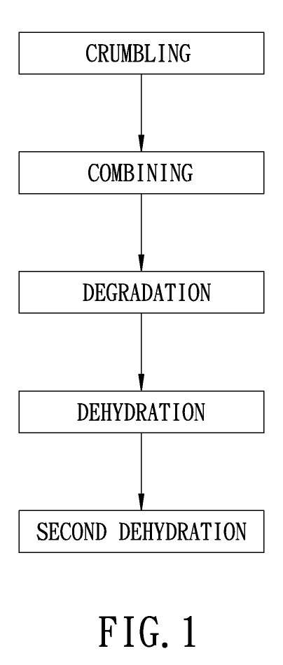

FIG. 1 shows a practicable flowchart for making a reducing sugar solution. A method for making reducing sugar comprising steps of:

Crumbling: the bio-substance containing lignocellulose is firstly crumbled, so that every particle of the bio-substance containing lignocellulose is smaller than 5 mm. Wherein, the bio-substance containing lignocellulose adopts hardwood, cork, grass, bamboo, aquatic species like algae and water hyacinth, or agricultural and industrial waste containing lignocellulose.

Combining: the bio-substance containing lignocellulose is combined with a solvent. Wherein, the solvent adopts water, and a combining ratio of the bio-substance containing lignocellulose divided by the water ranges from 0.1 to 1.0. Concurrently, the solvent further includes a catalyst, and a combining ratio of the catalyst divided by the bio-substance containing lignocellulose ranges from 0.5% to 10%. In this embodiment, the catalyst adopts sulfuric acid, nitric acid, or hydrochloric acid.

Degradation: conduct a degradation reaction on the bio-substance containing lignocellulose that is mixed with the solvent and placed in an autoclave. Wherein, a temperature in the degradation reaction within the autoclave ranges between 100 and 200° C. A heating rate before reaching the temperature for the degradation reaction ranges from 1 to 20° C./min. A reacting time after reaching the temperature for the degradation reaction ranges from 15 minutes to 5 hours.

Dehydration: a product generated from the degradation reaction is treated by centrifugal dehydration for separating the product into the reducing sugar solution and solid bio-remains. Wherein, a concentration of the reducing sugar solution ranges from 3 to 113.8 g/L.

Second Dehydration: the solid bio-remains are able to be combined with water and be further treated by another centrifugal dehydration for separating residual reducing sugar therefrom. Wherein, the water added in the second dehydrating is less than the previous dehydration. Therefore, the concentration of the reducing sugar solution in the second dehydration would be similar to the reducing sugar solution generated in the previous dehydrating.

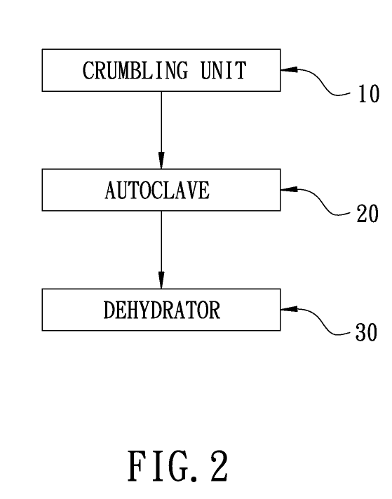

FIG. 2 shows a device in accordance with the present invention. A device for making reducing sugar comprises:

a crumbling unit 10 for crumbling the bio-substance containing lignocellulose, so that every particle of the bio-substance containing lignocellulose is smaller than 5 mm; in this embodiment, the crumbling unit 10 adopts a roller;

an autoclave 20 for accommodating the bio-substance containing lignocellulose and the solvent for inducing the degradation reaction of the bio-substance containing lignocellulose under a high temperature and a high pressure; wherein, a heating rate before reaching the temperature for the degradation reaction in the autoclave 20 ranges from 1 to 20° C./min; the temperature for the degradation reaction ranges between 100 and 200° C.; thereby, a reacting time after reaching the temperature for the degradation reaction ranges from 15 minutes to 5 hours;

a dehydrator 30 for dehydrating a product resulted from the degradation reaction so as to separate a reducing sugar solution of a high concentration from solid bio-remains; in this embodiment, the dehydrator 30 adopts a centrifugal dehydrator.

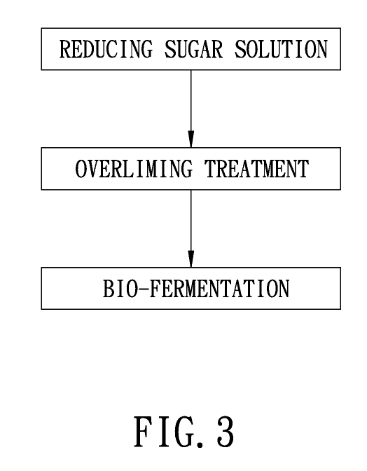

FIG. 3 shows a flowchart for treating the reducing sugar solution. Afore method and device for making the reducing sugar is convenient since users could simply crumble the bio-substance containing lignocellulose via the crumbling unit 10. After that, mix the crumbled bio-substance with the solvent and the catalyst well and place the mixture into the autoclave 20. Accordingly, the degradation reaction is conducted under a high temperature and a high pressure. While centrifugally dehydrating the mixture via the dehydrator 30, the reducing sugar solution of a high concentration is achieved. The following table shows the experimental data in accordance with the present invention:

TABLE 1

| | CATALYST: SULFURIC ACID/AMOUNT: 3%/DEGRADATION | | | | | |
|---|---|---|---|---|---|---|
| CONDITION | TEMPERATURE: 150° C./DEGRADATION TIME: 1 HR | | | | | |
| BIO-SUBSTANCE | GRASS | CORNSTALK | DISPOSABLE | RICE | RICE HUSK | RICE HUSK |
| | | | CHOPSTICKS | HUSK | | |
| | | | (BAMBOO) | | | |
| BIO-SUBSTANCE/ | 0.4 | 0.4 | 0.4 | 0.4 | 0.6 | 0.8 |
| SOLVENT | | | | | | |
| CONCENTRATION | 58.9 | 113.8 | 66.5 | 67.8 | 87.1 | 90.6 |
| OF THE | | | | | | |
| REDUCING SUGAR | | | | | | |

| | | | | | | |
|---|---|---|---|---|---|---|
| BIO-SUBSTANCE | RICE HUSK | ALGAE | BAGASSE | SAW DUSK | COTTON | FIBC BAG |
| | | POWDER | | | | |
| BIO-SUBSTANCE/ | 1 | 0.6 | 0.4 | 0.4 | 0.4 | 0.4 |
| SOLVENT | | | | | | |
| CONCENTRATION | 102.6 | 5.2 | 20.2 | 64.1 | 10.3 | 17 |
| OF THE | | | | | | |
| REDUCING SUGAR | | | | | | |

| | | | | |
|---|---|---|---|---|
| BIO-SUBSTANCE | JATROPHA | JATROPHA | DEGREASING | EXTRACTED THE |
| | CURCAS | CURCAS | JATROPHA | DEGREASING JATROPHA |
| | HULL | HULL | CURCAS SEED | CURCAS SEED AND THE HULL |
| | | | | THEREOF |
| BIO-SUBSTANCE/ | 0.4 | 1 | 0.4 | 0.4 |
| SOLVENT | | | | |
| CONCENTRATION | 9.9 | 18.3 | 15.8 | 30.5 |
| OF THE | | | | |
| REDUCING SUGAR | | | | |

It is evidently shown in Table 1 that the method and the device for making the reducing sugar in accordance with the present invention is able to produce the reducing sugar solution of a high concentration up to 113.8 g/L. Afore production could be further applied to fermentation so as to produce bio-ethanol, bio-butanol, lactic acid (which can be further processed to become a polylactic acid resin for bio-degradable plastic, methane, and bio-hydrogen. Particularly, before fermentation, the reducing sugar solution could be treated by overliming for removing bio-substance that is unbenefited to the fermentation. Thereby, the efficiency of the fermentation is promoted, which concurrently allows the bio-substance to solvent ratio of the reducing sugar solution to be enhanced in time of producing. As a result, the reducing sugar solution of a high concentration is achieved. Moreover, the bio-substance containing lignocellulose does not need the steaming through a high temperature after the degradation is conducted, so, not like the conventional technique, the present invention is benefit for reducing water amount and decreasing energy requirements. Further, the crumbling unit 10, the autoclave 20 and the dehydrator 30 are common apparatus in the market. Therefore, the present invention preferably is advantageous to acquire the apparatus, decrease the producing cost, and achieve an easy promotion.

I claim:

1. A method for making reducing sugar comprising steps of combining a bio-substance containing lignocellulose with a solvent, and conducting a degradation reaction via an autoclave for acquiring a reducing sugar solution of a high concentration.

2. The method as claimed in claim 1, wherein, said bio-substance containing lignocellulose adopts either hardwood, cork, grass, bamboo, aquatic species, or agricultural and industrial waste containing lignocellulose.

3. The method as claimed in claim 1, wherein, said solvent adopts water, and a combining ratio of said bio-substance containing lignocellulose divided by said water ranges from 0.1 to 1.0.

4. The method as claimed in claim 1, wherein, said solvent further includes an addition of a catalyst, and a combining ratio of said catalyst divided by said bio-substance containing lignocellulose ranges from 0.5% to 10%.

5. The method as claimed in claim 4, wherein, said catalyst adopts either sulfuric acid, nitric acid, or hydrochloric acid.

6. The method as claimed in claim 1, wherein, a temperature for conducting said degradation reaction ranges between 100° C. and 200° C.

7. The method as claimed in claim 6, wherein, a heating rate before reaching said temperature for said degradation reaction ranges from 1 to 20° C./min.

8. The method as claimed in claim 6, wherein, a reacting time after reaching said temperature for said degradation reaction ranges from 15 minutes to 5 hours.

9. The method as claimed in claim 1, wherein, a concentration of said reducing sugar solution ranges from 3 to 113.8 g/L.

10. The method as claimed in claim 1, wherein, said bio-substance containing lignocellulose is firstly crumbled, so that every particle of said bio-substance containing lignocellulose is smaller than 5 mm.

11. The method as claimed in claim 1, wherein, a product generated from said degradation reaction is treated by centrifugal dehydration for separating said product into said reducing sugar solution and solid bio-remains.

12. The method as claimed in claim 11, wherein, said solid bio-remains are able to be combined with water and be further treated by said centrifugal dehydration for separating residual reducing sugar therefrom.

13. The method as claimed in claim 1, wherein, said reducing sugar solution is able to be applied to bio-fermentation for producing bio-ethanol, bio-butanol, lactic acid, methane, and bio-hydrogen.

14. The method as claimed in claim 13, wherein, said reducing sugar solution could be firstly practiced by an Overliming Treatment for removing bio-substance that is unbeneficial for said bio-fermentation, thereby promoting a bio-fermentation effect.

15. A device for making reducing sugar comprising:
an autoclave for accommodating a bio-substance containing lignocellulose and a solvent and for inducing a degradation reaction of said bio-substance containing lignocellulose under a high temperature and a high pressure; and
a dehydrator for dehydrating a product resulted from said degradation reaction so as to separate a reducing sugar solution of a high concentration from solid bio-remains.

16. The device as claimed in claim 15, wherein, said device further comprises a crumbling unit for crumbling said bio-substance containing lignocellulose, so that every particle of said bio-substance containing lignocellulose is smaller than 5 mm.

17. The device as claimed in claim 15, wherein, said solvent adopts water, and a combining ratio of said bio-substance containing lignocellulose divided by said water ranges from 0.1 to 1.0.

18. The device as claimed in claim 15, wherein, said solvent further includes a catalyst, and a combining ratio of said catalyst divided by said bio-substance containing lignocellulose ranges from 0.5% to 10%.

19. The device as claimed in claim 18, wherein, said catalyst adopts either sulfuric acid, nitric acid, or hydrochloric acid.

20. The device as claimed in claim 15, wherein, a temperature for said degradation reaction within said autoclave ranges between 100 and 200° C.

21. The device as claimed in claim 15, wherein, a heating speed in said autoclave ranges from 1 to 20° C./min.

22. The device as claimed in claim 15, wherein, a reacting time after reaching said temperature for said degradation reaction ranges from 15 minutes to 5 hours.

23. The device as claimed in claim 15, wherein, said solid bio-remains are able to be mixed with water and dehydrated by said dehydrator, thereby separating residual reducing sugar from said solid bio-remains.
